(19) United States
(12) Patent Application Publication
(10) Pub. No.: US 2023/0094263 A1
(43) Pub. Date: Mar. 30, 2023

(54) Methods For Treating Coronavirus Disease

(71) Applicant: Nisha Cell Therapeutics Ltd., Mill Hill (GB)

(72) Inventor: Behnam Sadeghi, Stockholm (SE)

(21) Appl. No.: 17/997,680

(22) PCT Filed: May 3, 2021
(86) PCT No.: PCT/IB2021/053694

§ 371 Date: Nov. 1, 2022

Related U.S. Application Data
(60) Provisional application No. 63/020,532, filed on May 5, 2020.
(60) Provisional application No. 63/146,439, filed on Feb. 5, 2021.

(51) Int. Cl. A61K 35/28; A61K 35/19; A61K 35/16; A61K 38/38; A61K 31/7052; A61K 31/546; A61K 31/573; A61K 31/727; A61K 31/4706; A61K 31/407; A61K 38/21; A61K 31/513; A61K 31/5383; A61K 31/192; A61P 31/14

(52) U.S. Cl. CPC A61K 35/28; A61K 31/192; A61K 31/407; A61K 31/513; A61K 31/546; A61K 31/573; A61K 31/727; A61K 31/4706; A61K 31/5383; A61K 31/7052; A61K 35/16; A61K 35/19; A61K 38/38; A61K 38/215; A61P 31/14

(57) ABSTRACT

Provided herein are methods for treating coronavirus diseases. In one embodiment, the method comprises administering to a coronavirus disease patient a therapeutic amount of decidua stromal cells. In one embodiment, the method comprises administering to the subject a therapeutic amount of decidua stromal cells (DSCs). In some embodiments, the coronavirus disease is SARS, MERS or COVID-19. The method of the disclosure can be used to treat a subject who has acute respiratory distress syndrome (ARDS), acute lung injury (ALI), or both.

### RELATED APPLICATIONS

This application claims the benefit of U.S. Provisional Application No. 63/020,532, filed on May 5, 2020, and claims the benefit of U.S. Provisional Application No. 63/146,439, filed on Feb. 5, 2021. The entire teachings of the above applications are incorporated herein by reference.

### BACKGROUND

The recent COVID-19 pandemic caused by the new coronavirus, SARS-CoV-2, poses a serious global health emergency. The leading cause of mortality in severe COVID-19 patients is respiratory failure resulted from acute respiratory distress syndrome (ARDS), in which severe infection of coronavirus induces hyperinflammation characterized by fatal cytokine storm and immune cell invasion in lung. There is, as yet, no FDA-approved treatment for COVID-19 ARDS, other than placing patients on a ventilator. Therefore, there is an urgent need to develop new treatment for COVID-19 ARDS.

### SUMMARY

In one aspect, the present disclosure provides a method for treating a coronavirus disease in a subject in need thereof. In one embodiment, the method comprises administering to the subject a therapeutic amount of decidua stromal cells (DSCs).

In some embodiments, the coronavirus disease is SARS, MERS or COVID-19. In some embodiments, the coronavirus is COVID-19.

In some embodiments, the subject has acute respiratory distress syndrome (ARDS).

In some embodiments, the DSCs are infused to the subject intravenously. In some embodiments, the DSCs are infused to the subject within 1 to 20 days after initiation of ARDS in the subject. In some embodiments, the DSCs are infused to the subject within 72 hours after initiation of ARDS in the subject. In some embodiments, the DSCs are administered to the subject once every three days. In some embodiments, the DCSs are administered to the subject at a dose of 0.5-2×10⁶ cells/kg body weight of the subject. In some embodiments, the DCSs are administered to the subject at a dose of 1-1.2 ×10⁶ cells/kg body weight of the subject.

In some embodiments, the DSCs are suspended in saline with 5% human albumin. In some embodiments, the DCSs are suspended at 2-5 ×10⁶ cells/ml. In some embodiments, the DCSs are suspended at 2 ×10⁶ cells/ml.

In some embodiments, the DSCs are prepared from a placenta. In some embodiments, the DSCs are prepared from fetal membranes of the placenta. In some embodiments, the DSCs express CD29, CD73, CD90, CD105, CD49d, CD44, CD54, HLA class I, PD-L1 and PD-L2. In some embodiments, the DSCs do not express CD45, CD34, CD31 or HLA class II. In some embodiments, the DSCs do not express CD11b, CD19, CD45, CD34, CD31 or HLA class II.

In some embodiments, the DSCs are allogeneic to the subject.

In some embodiments, the subject is a human patient between about 6 months and 75 years old.

In another aspect, the present disclosure provides a method for treating a viral-induced ARDS in a subject in need thereof, the method comprising administering to the subject a therapeutically effective amount of DSCs.

In some embodiments, the viral is a coronavirus.

In some embodiments, the coronavirus is SARS, MERS or COVID-19.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows the cell surface phenotype of the isolated and expanded DSCs, characterized by flow cytometry. DSCs were positive for the typical MSC markers CD29, CD73, CD90, and CD105 but negative for hematopoietic markers CD11b, CD34 and CD45.

FIG. 2A compares the opacities and infiltration in the lungs 2 days before DSC therapy and 4 days after the first dose of DSC therapy. FIG. 2B compares the opacities and infiltration in the lungs 2 days before DSC therapy and 9 days after the first dose of DSC therapy (2 days after the second dose of DSC therapy).

FIGS. 3A-3D show that DSC infusion decrease the level of IL-6 (A), CRP (B), G-CSF (C) and CCL-2 (D).

FIG. 4 shows the dynamism of blood oxygen level before and after DSC infusion.

FIG. 5 summarizes patients' characteristics. *: Despite achieving a partial response to the DSC therapy, this patient stopped taking medications in the middle of the treatment course on his own initiative. The patient left the hospital against recommendations by the physicians and staff and accepted the responsibility and risk on his own.

FIG. 6 presents cytokines and oxygen saturation in each patient before and after the DSC therapy. Base = one day before DSC therapy. After = one day after the last DSC infusion or on the day of discharge.

### DETAILED DESCRIPTION

A description of example embodiments follows.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In one aspect, the present disclosure provides a method for treating a coronavirus disease in a subject in need thereof. In one embodiment, the method comprises administering to the subject a therapeutic amount of decidua stromal cells (DSCs). In some embodiments, the coronavirus disease is SARS, MERS or COVID-19. In some embodiments, the coronavirus is COVID-19. In some embodiments, the subject has acute respiratory distress syndrome (ARDS).

In another aspect, the present disclosure provides a method for treating a viral-induced ARDS in a subject in need thereof, the method comprising administering to the subject a therapeutically effective amount of DSCs. In some embodiments, the viral is a coronavirus. In some embodiments, the coronavirus is SARS, MERS or COVID-19.

### Definitions

As used herein, the singular forms “a”, “an” and “the” include plural references unless the context clearly dictates otherwise.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

The term “about” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger. Unless explicitly stated otherwise, all of the numbers and ranges provided in the application are qualified by the term “about.”

Also, unless expressly specified or otherwise required by context (e.g., in some Examples), all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameter.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

As used herein, the term “coronavirus’ includes any member of the family Coronaviridae, including, but not limited to, any member of the genus Coronavirus, and any member of the genus Torovirus. The term “coronavirus” further includes naturally-occurring (e.g., wild-type) coronavirus; naturally-occurring coronavirus variants; and coronavirus variants generated in the laboratory, including variants generated by selection, variants generated by chemical modification, and genetically modified variants (e.g., coronavirus modified in a laboratory by recombinant DNA methods).

The term “subject” or “individual” or “animal” or “patient” as used interchangeably herein refers to human in need of diagnosis, prognosis, amelioration, prevention and/or treatment of a disease or disorder such as infection of coronavirus. In some embodiments, the subject has developed symptoms of coronavirus (e.g., COVID-19 induced ARDS). In some embodiments, the subject has a risk of developing symptoms of coronavirus (e.g., COVID-19 induced ARDS).

As used herein, a “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

### Decidual Stromal Cells

The human decidua is a specialized tissue characterized by embryo-receptive properties. The success of human pregnancy strongly depends on embryo quality and the physiological state of the uterine lining-an epithelial tissue layer called endometrium. To prepare the uterus for embryo implantation and pregnancy, the endometrium undergoes a process termed decidualization. During this process, the endometrial epithelium, blood vessels, and stroma are transformed into a specialized tissue, called decidua, which is composed of glands, immune cells, blood and lymph vessels, and decidual stromal cells (DSCs).

The decidualization process initiates during the midsecretory phase of the menstrual cycles as a result of the elevated levels of ovarian hormones — oestrogen and progesterone —independent of the presence of an implanting blastocyst. The decidualization process causes a gradual and profound alteration in gene expression, cellular functions, and tissue remodeling until the complete formation of a placenta during pregnancy. Analyses of gene expression and secretome of the decidua reveal changed profiles of signal messengers/intermediates, transcription factors, hormones/growth factors, cytokines, chemokines, adhesion molecules, ligands/receptors, cytoskeleton organization, composition of extracellular matrix, ion and water transport, cell cycle regulation, cell trafficking, migration and functions, angiogenesis, decidual receptivity, and implantation.

Decidua stromal cells (DSCs), which constitute the main cellular component of human decidua, fetal membranes or placenta, play an important role in the embryo implantation, development of pregnancy, and maternal-fetal immune tolerance. Notably, DSCs, when isolated from human placenta and/or fetal membranes, demonstrate properties that distinguish them from mesenchymal stromal cells (MSCs) from other organs like bone marrow. For example, DSCs induce FOXP3-positive regulatory T cells and inhibit alloreactivity in vitro in a contract-dependent manner and not only by soluble factors like MSCs. DCSs are half the size of MSCs and do not differentiate well to chondrocytes and osteocytes. In the allogeneic setting, DSCs promote an anti-inflammatory cytokine profile. DSCs also have stronger hemostatic properties than MSCs. DSCs have typical MSCs surface markers, but a stronger expression of programmed death-ligand 1(PD-L1) and PD-L2, and CD49d than MSCs from bone marrow.

The DSCs of the present disclosure can be prepared from human term placentas as described earlier (see, e.g., H Karlsson et al., Clin Exp Immunol, (2012) 167:543-55). For example, the DSC can be isolated from the placenta after caesarian section and/or placenta from normal delivery. In some embodiments, the DSCs are isolated from one or more placenta after caesarian section. In short, to prepare the DCSs, the fetal membranes are dissected from the placenta. DSCs can be isolated from Placenta or combination of placenta and fetal membranes. Single-cell suspension can be made by treating the dissected fetal membranes with a digestive enzyme and/or physical disruption, a non-limiting example of which is mincing and flushing the tissue parts through a nylon filter or by gentle pipetting with washing medium. Single-cell suspension can also be made by treating dissected pieces of placenta with a digestive enzyme and/or physical disruption.

The DCSs of the present disclosure may be obtained, in various embodiments, from a full-term or pre-term placenta. Optionally, residual blood is removed from the placenta before cell harvest. This may be done by a variety of methods known to those skilled in the art, for example by perfusion, i.e., pouring or passaging a fluid over or through the placental tissue. In certain embodiments, the placental tissue may be from any mammal, while in other embodiments, the placental tissue is human. A convenient source of placental tissue is a post-partum placenta (e.g., less than 48 hours after birth or less than 48 hours after birth), however, a variety of sources of placental tissue or cells may be contemplated by the skilled person. In some embodiments, the placenta is used within 48 hours, within 47 hours, within 46 hours, within 45 hours, within 44 hours, within 43 hours, within 42 hours, within 41 hours, within 40 hours, within 39 hours, within 38 hours, within 37 hours, within 36 hours, within 35 hours, within 34 hours, within 33 hours, within 32 hours, within 31 hours, within 30 hours, within 29 hours, within 28 hours, within 27 hours, within 26 hours, within 25 hours, within 24 hours, within 23 hours, within 22 hours, within 21 hours, within 20 hours, within 19 hours, within 18 hours, within 17 hours, within 16 hours, within 15 hours, within 14 hours, within 13 hours, within 12 hours, within 11 hours, within 10 hours, within 9 hours, within 8 hours, within 7 hours, within 6 hours, within 5 hours, within 4 hours, within 3 hours, within 2 hours, or within 1 hour of birth. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

The DSCs isolated from the placenta are subsequently cultured and propagated in vitro, e.g., in a cell culture medium (e.g., DMEM or alfa MEM, RPMI or any other sterile culture media) comprising 2-20% (e.g., 10%) fetal calf serum (FCS), 2-20% (e.g., 10%) fetal bovine serum (FBS), or 2-20% (e.g., 5%) human platelet lysate. In some embodiments, the DSCs isolated from the placenta are subsequently cultured and propagated in vitro, e.g., in DMEM with 10% fetal calf serum or in a cell culture medium (e.g., DMEM or alfa MEM) comprising 2-20% (e.g., 10%) fetal calf serum, 2-20% (e.g., 10%) human platelet lysate or 2-20% (e.g., 5%) human platelet lysate. In some embodiments, the DSCs isolated from the placenta are subsequently cultured and propagated in DMEM with 10% fetal calf serum or in a cell culture medium (e.g., DMEM or alfa MEM) comprising 2-20% (e.g., 10%) fetal calf serum, 2-20% (e.g., 10%) human platelet lysate or 2-20% (e.g., 5%) human platelet lysate. In specific embodiments, the DSCs isolated from the placenta are subsequently cultured and propagated in DMEM with 10% fetal calf serum. In some embodiments, the DSCs are suspended in saline with about 2.5-10% human plasma, human albumin or human platelet lysate. In some embodiments, the DSCs are suspended in saline with about 5% human albumin. In some embodiments, the DSCs can be cultured in a combination of 2D and 3D culturing conditions (e.g., in a bioreactor, a multilayer flask, or a bead based system).

Those skilled in the art will appreciate in light of the present disclosure that the cells extracted from a placenta, may be, in some embodiments, followed by marker-based cell sorting before being subjected to the culturing methods described herein. The DSCs of the present disclosure express CD29, CD73, CD90, CD105, CD49d, CD44, CD54, HLA class I, PD-L1 and PD-L2. The DSCs of the present disclosure are negative for the hematopoietic markers CD11b, CD19, CD45 and CD34, the endothelial marker CD31, and HLA calls II.

In some embodiments, the DSCs:
a) express CD29, CD73, CD90, CD105, PDL-1, PDL-2;
b) do not express CD11b, CD45, CD34; or
c) both a) and b).

In some embodiments, the DSCs:
a) express CD29, CD73, CD90, CD105;
b) do not express CD11b, CD45, CD34; or
c) both a) and b).

Isolated DCSs of the present disclosure can be stored frozen in liquid nitrogen until use. In certain embodiments, the isolated DSCs are suspended before use in saline with 5% human plasma or human albumin. In some embodiments, the isolated DSCs are suspended before use in saline with 2-20% (e.g., 5%) human plasma, human serum albumin or 2-20% (e.g., 5%) human platelet lysate. In some embodiments, the isolated DSCs are suspended before use in saline with 2-20% (e.g., 5%) human plasma or human serum albumin. In some embodiments, the DCSs are suspended at 1-5×10⁶ cells/ml, e.g., about: 1-4.S×10⁶ cells/ml, 1.5-4.5×10⁶ cells/ml, 1.5-4×10⁶ cells/ml, 2-4x10⁶ cells/ml, 2.5-3.5×10⁶ cells/ml or 2.5-3×10⁶ cells/ml. In some embodiments, the DCSs are suspended at 1-2 ×10⁶ cells/ml. In some embodiments, the DSCs of the present disclosure are suspended at about 1×10⁶ cells/ml, 1.1×10⁶ cells/ml, 1.2×10⁶ cells/ml, 1.3×10⁶ cells/ml, 1.4×10⁶ cells/ml, 1.5×10⁶ cells/ml, 1.6×10⁶ cells/ml, 1.7×10⁶ cells/ml, 1.8×10⁶ cells/ml, 1.9x10⁶ cells/ml, 2.0×10⁶ cells/ml, 2.1×10⁶ cells/ml, 2.2×10⁶ cells/ml, 2.3×10⁶ cells/ml, 2.4×10⁶ cells/ml, or 2.5×10⁶ cells/ml. In certain embodiments, the DSCs of the present disclosure are suspended at about 1×10⁶ cells/ml, 1.1x10⁶ cells/ml, 1.2×10⁶ cells/ml, 1.3×10⁶ cells/ml, 1.4×10⁶ cells/ml, 1.5×10⁶ cells/ml, 1.6×10⁶ cells/ml, 1.7×10⁶ cells/ml, 1.8×10⁶ cells/ml, 1.9×10⁶ cells/ml, 2.0×10⁶ cells/ml, 2.1×10⁶ cells/ml, 2.2×10⁶ cells/ml, 2.3×10⁶ cells/ml, 2.4×10⁶ cells/ml, 2.5×10⁶ cells/ml, 2.6×10⁶ cells/ml, 2.7×10⁶ cells/ml, 2.8×10⁶ cells/ml, 2.9×10⁶ cells/ml, 2.1×10⁶ cells/ml, 2.2×10⁶ cells/ml, 2.3×10⁶ cells/ml, 2.4×10⁶ cells/ml, 2.5×10⁶ cells/ml, 2.6×10⁶ cells/ml, 2.7×10⁶ cells/ml, 2.8×10⁶ cells/ml, 2.9×10⁶ cells/ml, 3.0×10⁶ cells/ml, 3.1x10⁶ cells/ml, 3.2×10⁶ cells/ml, 3.3×10⁶ cells/ml, 3.4×10⁶ cells/ml, 3.5×10⁶ cells/ml, 3.6×10⁶ cells/ml, 3.7×10⁶ cells/ml, 3.8×10⁶ cells/ml, 3.9×10⁶ cells/ml, 4.0×10⁶ cells/ml, 4.1×10⁶ cells/ml, 4.2×10⁶ cells/ml, 4.3×10⁶ cells/ml, 4.4×10⁶ cells/ml, 4.5×10⁶ cells/ml, 4.6×10⁶ cells/ml, 4.7×10⁶ cells/ml, 4.8x10⁶ cells/ml, 4.9×10⁶ cells/ml or 5×10⁶ cells/ml.

In some embodiments, the DSCs comprises cells from Passage 2, 3, 4, 5, 6, 7, 8, 9 or 10, or a combination thereof.

In some embodiments, the subject is a human patient between about 6 months and 75 years old, for example, 2-75 years old, 2-70 years old, 5-70 years old, 5-65 years old, 10-65 years old, 10-60 years old, 15-60 years old or 15-55 years old.

### Coronavirus Disease

Coronaviruses are a group of related viruses that cause diseases in mammals and birds. In humans, coronaviruses cause respiratory tract infections that can range from mild to lethal. Mild illnesses include common cold, while more lethal case can cause SARS, MERS and COVID-19.

Coronaviruses belong to the subfamily Orthocoronavirinae, in the family Coronaviridae, order Nidovirales, and realm Riboviria. Coronaviruses are enveloped viruses with a positive-sense single-stranded RNA genome and a nucleocapsid of helical symmetry. The genome size of coronaviruses ranges from approximately 26 to 32 kilobases. Coronaviruses have characteristic club-shaped spikes that project from their surface, which in electron micrographs create an image reminiscent of the solar corona from which their name derives.

Six species of human coronaviruses are known, with one species subdivided into two different strains, making seven strains of human coronaviruses altogether. Four of these coronaviruses continually circulate in the human population and produce the generally mild symptoms of the common cold worldwide. These four mild coronaviruses are: human coronavirus OC43 (HCoV-OC43), HCoV-HKU1, HCoV-229E, HCoV-NL63. Three human coronaviruses produce symptoms that are potentially severe: SARS-CoV, MERS-CoV and SARS-CoV-2.

Severe acute respiratory syndrome-related coronavirus (SARS-CoV, or SARS-CoV-1) is identified as the cause of the severe acute respiratory syndrome (SARS) outbreak during 2002-2004. Symptoms of SARS are flu-like and may include fever, muscle pain, lethargy, cough, sore throat, and other nonspecific symptoms. SARS may eventually lead to shortness of breath and pneumonia.

Middle East respiratory syndrome (MERS) is caused by the infection of MERS-CoV. Symptoms of MERS may range from non, to mild, to server. Typical symptoms include fever, cough, diarrhea, and shortness of breath.

The current COVID-19 pandemic is caused by the SARS-CoV-2. Common symptoms of COVID-19 include fever, cough and shortness of breath. Other symptoms may include fatigue, muscle pain, diarrhea, sore throat, loss of smell, and abdominal pain. While the majority of cases result in mild symptoms, some progress to viral pneumonia and multi-organ failure. The leading cause of mortality in severe COVID-19 patients is respiratory failure resulted from acute respiratory distress syndrome (ARDS).

ARDS is a type of respiratory failure characterized by rapid onset of widespread inflammation in the lungs. Symptoms of ARDS include shortness of breath, rapid breathing, and bluish skin coloration. ARDS may be caused by sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body’s regulation of blood clotting. In effect, ARDS impairs the lungs' ability to exchange oxygen and carbon dioxide. Adult diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O.

### Methods of Treatment

In one aspect, the present disclosure provides methods for treating a coronavirus disease in a subject in need thereof. In certain embodiments, the method comprises administering to the subject a therapeutic amount (e.g., number) of DSCs as described herein. In some embodiments, the administration of DSCs ameliorates the acute respiratory distress syndrome (ARDS) in the subject.

In certain embodiments, the DSCs are administered to the subject in a dose of 1-2 ×10⁶ cells per kg body weight of the subject, i.e., 1 ×10⁶ cells/kg, 1.1x10⁶ cells/kg, 1.2×10⁶ cells/kg, 1.3×10⁶ cells/kg, 1.4×10⁶ cells/kg, 1.5×10⁶ cells/kg, 1.6×10⁶ cells/kg, 1.7×10⁶ cells/kg, 1.8×10⁶ cells/kg, 1.9×10⁶ cells/kg, 2×10⁶ cells/kg.

In certain embodiments, the DSCs are administered to the subject in a dose of 0.5-10 ×10⁶ cells per kg body weight of the subject, e.g., about: 0.6×10⁶ cells/kg, 0.7×10⁶ cells/kg, 0.8×10⁶ cells/kg, 0.9×10⁶ cells/kg, 1 ×10⁶ cells/kg, 1.1x10⁶ cells/kg, 1.2×10⁶ cells/kg, 1.3×10⁶ cells/kg, 1.4×10⁶ cells/kg, 1.5×10⁶ cells/kg, 1.6×10⁶ cells/kg, 1.7×10⁶ cells/kg, 1.8×10⁶ cells/kg, 1.9×10⁶ cells/kg, 2×10⁶ cells/kg, 2.1×10⁶ cells/kg, 2.2×10⁶ cells/kg, 2.3×10⁶ cells/kg, 2.4×10⁶ cells/kg, 2.5×10⁶ cells/kg, 3.0×10⁶ cells/kg, 3.5×10⁶ cells/kg, 4.0×10⁶ cells/kg, 4.5×10⁶ cells/kg, 5.0×10⁶ cells/kg, 5.5×10⁶ cells/kg, 6.0x10⁶ cells/kg, 6.5×10⁶ cells/kg, 7.0×10⁶ cells/kg, 7.5x10⁶ cells/kg, 8.0x10⁶ cells/kg, 8.5x10⁶ cells/kg, 9.0×10⁶ cells/kg, 9.5×10⁶ cells/kg, or 10.0×10⁶ cells/kg.

In some embodiments, the DSCs described herein are administered intramuscularly, intratracheally, intrathecal, intravenously, intraarterial, subcutaneously, or intraperitoneally. In certain embodiments, the DSCs described herein are administered intramuscularly, intratracheally, intrathecal, intravenously, subcutaneously, or intraperitoneally. In certain specific embodiments, the DSCs described herein are administered intravenously, subcutaneously, or intraperitoneally. In certain specific embodiments, the DSCs described herein are administered intravenously or intratracheally. In other embodiments, the DSCs are administered intramuscularly; while in other embodiments, the DSCs are administered systemically. In this regard, “intramuscular” administration refers to administration into the muscle tissue of a subject; “subcutaneous” administration refers to administration just below the skin; “intravenous” administration refers to administration into a vein of a subject; and “intraperitoneal” administration refers to administration into the peritoneum of a subject. “Intratracheal” administration refers to administration into the trachea of a subject. “Intrathecal” administration refers to administration into the spinal theca of a subject. “Intraarterial” administration refers to administration into the artery of a subject.

In certain embodiments, the DSCs to be administered to the subject is a mixture of cells of maternal origin and fetal origin. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells. In other embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1 %, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% fetal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96- 99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% fetal cells.

In certain embodiments, the DSCs has a cell viability of at least about 85%, for example, at least about: 86%, 87%, 88%, 89%, 90%, 91%, 92%, 93%, 94%, 95%, 96%, 97%, 98% or 99%. In certain embodiments, the DSCs has a cell viability of at least 90%.

In certain embodiments, the DSCs are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of the diagnosis or initiation of respiratory distress syndrome. In some embodiments, the DSCs are administered after the subject is stabilized from acute pathologies. In some embodiments, the subject is stabilized by supportive medical care. In some embodiments, the DSCs described herein are administered in about 1-20 days after initiation of ARDS, for example, in about: 1-18, 2-18, 2-16, 3-16, 3-14, 4-14, 4-12, 5-12, 5-10, 6-10 or 6-8 days. In specific embodiments, the DSCs described herein are administered 1-168, 2-168, 2-156, 3-156, 3-144, 4-144, 4-132, 5-132, 5-120, 6-120, 6-108, 7-108, 7-96, 8-96, 8-84, 9-84, 9-72, 10-72, 10-60, 12-60, 12-48, 14-48, 14-36, 16-36, 16-24, 18-24, 20-24, 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18- 72, 24-72, or 36-72 hours after the diagnosis or initiation of respiratory distress syndrome. In more specific embodiments, the DSCs described herein are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18- 72, 24-72, or 36-72 hours after the diagnosis or initiation of respiratory distress syndrome. In still other embodiments, the described compositions are administered 3-48, 4-48, 5-48, or 6-48 hours after the diagnosis of respiratory distress syndrome. In some embodiments, the DSCs are infused to the subject within about 1-20 days after initiation of ARDS in the subject. In some embodiments, the DSCs are infused to the subject within about 3-168 hours after initiation of ARDS in the subject.

In certain embodiments, the DSCs are administered to the subject repeatedly. In certain embodiments, the DSCs are administered to the subject once every 2-7 days, for example, every 2 days, every 3 days, every 4 days, every 5 days, every 6 days or every 7 days. In certain embodiments, two or more doses of the DSCs are administered to the subject once every three or four days. In certain embodiments, the DSCs are administered to the subject once every three days.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Exemplification

### Materials and Methods

### Patients

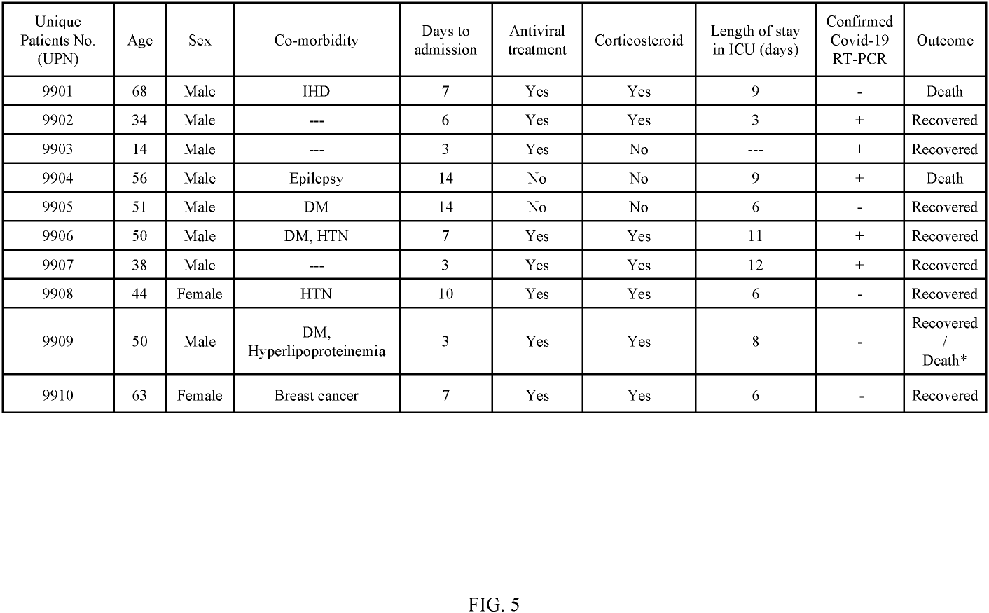

Between March until November 2020, ten ARDS patients following coronavirus infection (eight males and two females) were treated with decidua stromal cells (DSCs). The mean age of patients was 46.8 years (range of 14-68 years). Covid-19 infection was confirmed, by the quantitative reverse-transcriptase polymerase chain reaction (qRT- PCR) technique using kit specific for E and N genes of SARS-CoV-2 (OSANG Healthcare, South Korea) based on the manufacturer and WHO guideline on nasopharyngeal and oropharyngeal swab samples. Some patients did not have positive Covid-19 test in several assays (FIG. 5). Patients characteristics are shown in FIG. 5.

The ethical committee of Motamed Cancer Institute, Tehran, Iran approved the isolation of DSCs and applying DSC for the treatment of ARDS patients (ID No. IR.ACECR.IBCRC.REC.1395.13). The study also was registered in Iranian registry of clinical trial (IRCT2017010531786N1). All patients signed informed consent and for the child, his guardian agreed and signed the consent. None of the included patients were deprived of any available medications for Covid-19.

### Decidua Stromal Cells (DSCs) Isolation and Expansion

Isolation, expansion, and DSC preparation for injection was explained elsewhere but briefly, the fetal membranes were dissected from the placenta tissues, and underwent enzymatic digestion (See, e.g. Ringden, O., et al., Stem Cells Transl Med, 2018. 7(4): p. 325-331). DSCs were isolated and expanded under good manufacturing practice conditions in a laboratory equipped with laminar flow hood, sterile bench, and separate incubators for different batch of DSCs. Isolated DSCs were characterized by flowcytometry and were evaluated for karyotype analysis and the origin (they should be mother origin) and expanded until passage five (P5). Samples from the supernatant as well as infusion media were checked for sterility regularly. Subsequently DSC were frozen slowly in fetal bovine serum (FBS, GIBCO, Germany) containing 1-10% (e.g., 5%) dimethyl sulfoxide (DMSO, Sigma, Germany) or an optimized freeze media (e.g., Cryostor 5 (CS5) or Cryostor 10 (CS10), BioLife Solutions, Bothell, WA) and stored in liquid nitrogen until use. Upon the request from the ICU units and based on the patients' weight (1×10⁶ DSCs/kg body weight) appropriate number of vials were transferred to the GMP lab. Vials were thawed rapidly in an incubator/water bath (37° C.) and cell suspension was resuspended in infusion buffer, sodium chloride 0.9% (saline) buffer supplemented with 5% human serum albumin, (CSL, Behring, UK). The DSCs were washed two times with the infusion buffer and lastly cell suspension was passed through a 70-µM cell strainer (Corning, New York, US). Cell number and viability was calculated and if the cell viability was less than 90%, the batch was not qualified for infusion. Final DSC dose/infusion was calculated based on the patients' weight (1×10⁶ DSCs/kg).

DSC was resuspended in relevant volume of infusion buffer (sodium chloride 0.9% supplemented with 5% human serum albumin) at the concentration of 2×10⁶ DSCs/mL. Finally, cell suspension was transferred into a heparinized syringe (V-med, IRAN) and was shipped at room temperature in a biologic sample box to the hospital in less than 30 minutes.

### Cell Infusion Procedure

To prevent any possible allergic reaction, five to ten minutes before cell infusion each patient received either 10 mg of chlorphenamine (10 mg/ml) or 100 mg hydrocortisone, intravenously five to ten minutes before each cell infusion. All DSCs infusions was done via peripheral intravenous catheter (20-gauge, pink, bore 1 mm). Immediately before and after DSC infusion, the venous line was flushed with 5ml low molecular weight heparin (Caspian Tamin, IRAN) at the dose of 1000 IU/ml. If the patients need more than one dose, the next dose of DSC was infused three days after previous dose.

### Cytokine Measurement

At different time points, before and after DSC infusion, blood samples were collected from the patients. Blood was immediately centrifuged and serum was separated, labelled and immediately were frozen and stored at -80° C. freezer until analysis. ELISA was carried out to determine the serum levels of different cytokines including CCL2, IL-6, GCSF using R&D kits (R&D Systems, USA) according to the manufacturer instructions. A standard curve was derived from the serial dilutions for each cytokine. Finally, the absorbance of each well was determined by a microplate spectrophotometer (epoch 2, BioTek, USA) at 450 nm and the cytokine concentrations were estimated by referring to the standard curve. Due to sampling limitation and overloading services, it was not possible to measure cytokines in all patients.

### Flow Cytometry

To explore DSC phenotype cells were stained with the following antibodies according to manufacturer instruction: CD14, CD45, CD90, CD73, CD29, CD105 and CD11b (all from BD Bioscience, USA, San Diego, CA, USA). For flow cytometric analyses, a fluorescence activated cell sorter (FACS) BD FACS Calibur (BD biosciences, San Jose, CA, USA) was used to acquire and analysis data. In the patients' blood samples, the number of CD3⁺, CD4⁺ and CD8⁺ lymphocytes as well as CD4⁺/CD8⁺ ratio was evaluated using CD3⁺, CD4⁺, and CD8⁺, all from BD Bioscience (USA). Cells population and frequency was measured on gated lymphocyte population using flow cytometry (BD FACS Lyric, BD Bioscience, USA). CT scan

CT scan was performed with a commercial Computed Tomography (CT) Scanning using CT scanning System, SOMATOM Emotion eco (16-slice configuration) (SIEMENS, GERMANY). For the patients who were intubated, or unconscious CT scan could not be done rather chest X ray was done.

### Statistical Analysis

Patients’ results are shown as individual as well as cumulative data as median. To analysis before and after intervention (DSC treatment), for related samples Wilcoxon signed rank test was used. Pre-intervention data are related to the day of injection (before DSC infusion) and post-intervention data are related to the day after the last dose of DSC was infused. All statistical analyses were performed with the SPSS software version 22. P value less than 0.05 was considered as a statistically significant level.

### Results

### DSC Characterization

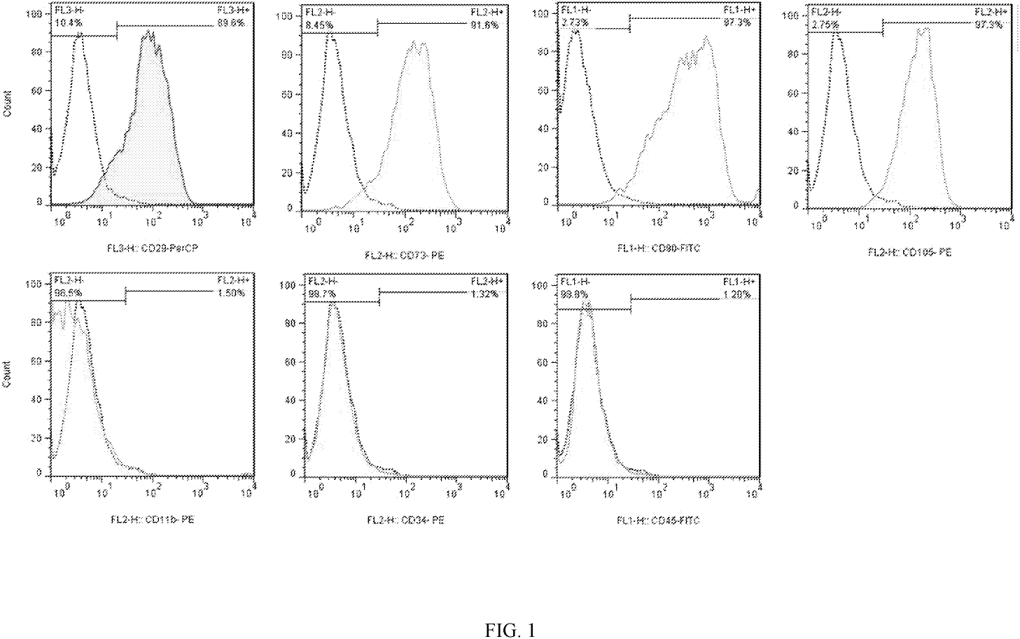

The cell surface phenotype of the isolated and expanded DSCs was characterized by flow cytometry. Like other studies DSCs were positive for the typical MSC markers CD29, CD73, CD90, and CD105 but negative for hematopoietic markers CD11b, CD34 and CD45 (FIG. 1). Furthermore, all DSCs batches were from maternal origin and chromosomal analysis showed normal karyotype (data not shown).

### Infusion Information and DSC Data

In the present study ten Covid-19 induced ARDS patients were treated with DSCs. On average each patient received 1.9 infusion (range of 1-3). The median DSC dose was 1.02x10⁶ cells/kg (range 0.85-1.23). In majority of infusion the DSCs were at Passage 4 (P4) and/or P5 (just in one infusion, DSC was at P3). The DSC viability at infusion time was always more than 90% (median 94%, range 90%-96%).

### Patients Outcome

### Patients 1 (UPN 9901)

A 68-year-old man with known (seven years) history of ischemic heart disease (IHD) was admitted to the emergency unit with a cough that started seven days ago, fever and shivering for three days. The patient was alert (Glasgow Coma Scale, GCS 15/15) but fatigue. In the CT scan ground glass opacities with distribution to peripheral was observed in more than 50% of the lung tissue. Saturation of peripheral oxygen (SpO2) was 88% at admission and the patient was given 8 L/min oxygen by face mask. Hydroxychloroquine, and several types of antibiotics started for the patient. LDH raised to 1300 iU/L and respiratory rate increased up to 21 /minutes and SpO2 dropped to 77% (without mask) despite patient receiving 8 L/min of oxygen. Fever, cough, and dyspnea became worse and five days after admission IVIG 25 g, IFN B-1 (12 million IU), Kaletra (Lopinavir/ritonavir) 200 mg (after six days) and hydrocortisone were instituted. While Oxygen consumption increased (10 L/min), oxygen saturation did not improve (88%) even with bilevel positive airway pressure (BiPAP).

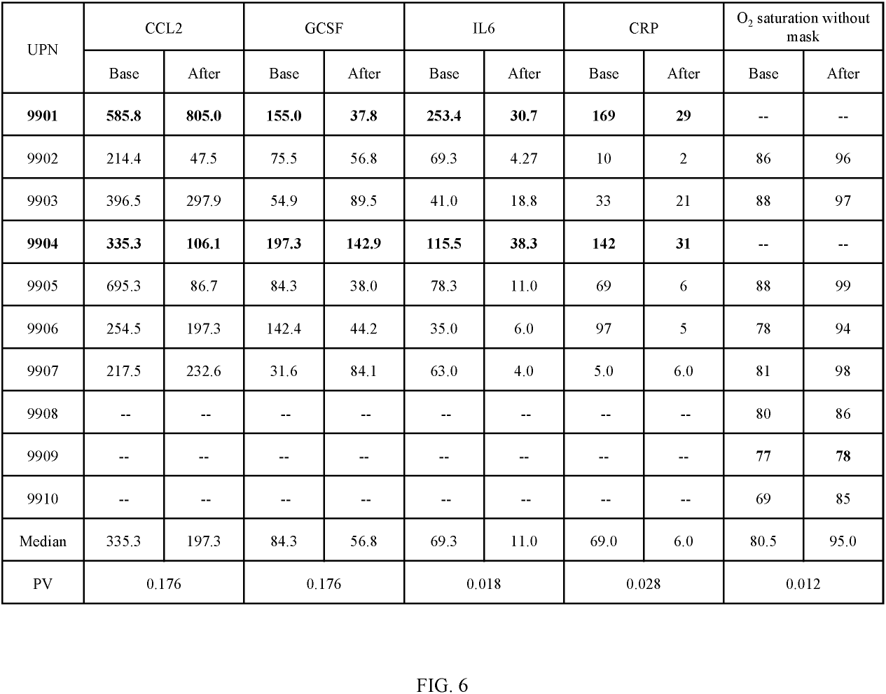

The following day, oxygen saturation dropped to 60%, the patient was intubated and connected to a ventilator. LDH reached to 1556 IU/L, and patient’s GCS dropped to 10/15. Due to the drop in oxygen saturation level as well as deteriorating of the patient’s situation DSC therapy was considered as the only option. Five days after admission, the first dose of DSC was infused (1x10^6 /kg body weight). Immediately after DSC infusion the SpO2 increased from 71% to 88%. After DSC infusion the oxygen saturation still was unstable but never below 88%. The second dose of DSC was infused two days after the first dose, but the patient did not improve and after 24 hours patient died due to cardiac arrest and multi organ failure. Following infusion of DSC there was decline in IL-6, G-CSF, CRP (FIG. 6).

### Patients 2 (UPN 9902)

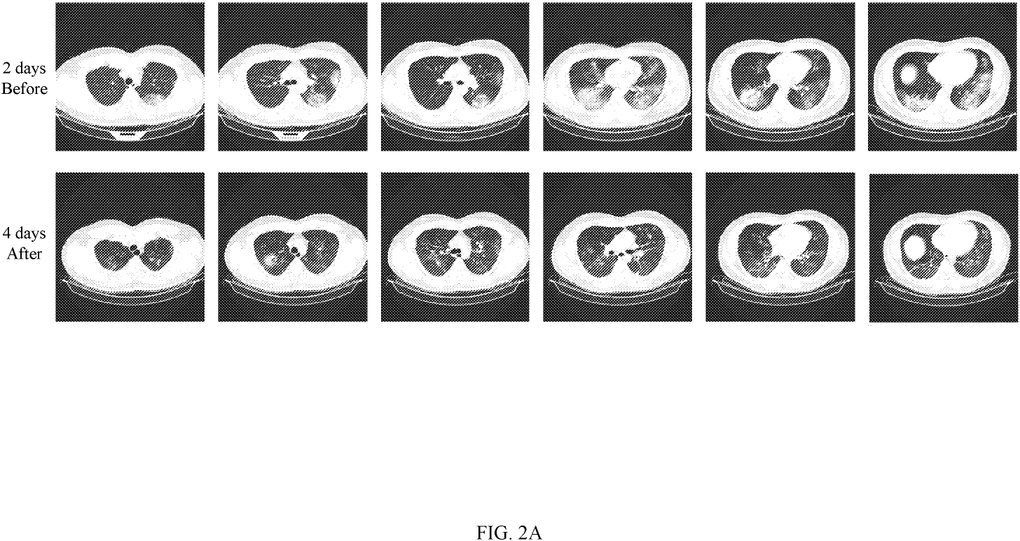

A 34-year-old man with no history of co-morbidity was admitted to the hospital with fever, shivering, night sweating, nausea, vomiting, and dyspnea started for six days. Oxygen saturation at admission was 86% and the patient was conscious (GCS 15/15). Oxygen supply at admission was 7 Lit/min via face mask. Hydroxychloroquine (400 mg/day), oseltamivir (75 mg/day), hydrocortisone (200 mg/day) naproxen and Azithromycin (250 mg/day) were instituted. CT scan showed ground glass opacities in more than 50% of both lungs concentrating in the middle lobe. Despite treatment patient developed severe dyspnea, could not walk, or even speak due to continuous dry cough, just sitting in the bed. The first dose of DSCs was infused two days after hospitalization and the second dose was given three days later (five days after hospitalization). IL-6, G-CSF, CRP, CCL2 dramatically decreased (FIG. 6). 24 hours after the first DSC dose, the patient felt less pressure on chest, breathing improved, frequency and intensity of coughing decreased in a way that the patient was able to speak with the medical staff. Six days after the second DSC dose, the patient was discharged from hospital, with the oxygen saturation of >96% (FIG. 6) and with good health and was suggested to stay home quarantine. As shown in FIG. 2A, opacities and infiltration in the middle and bottom of both lungs disappeared 4 days after the first dose of DSC therapy (FIG. 2A).

### Patients 3 (UPN 9903)

A 14-year-old boy admitted to the corona ward due to three days fever, cough, severe myalgia, abdominal pain, and sore throat started three days ago. In physical exam lymphadenopathy at neck and axillaries was observed. The patient had dyspnea, but oxygen saturation was 94% with 7 L/min oxygen by face mask. CT scan imaging showed unilateral ground glass opacity from center to periphery (FIG. 2B). In exploratory CT scan hepatomegaly, splenomegaly and significant mesenteric lymphadenopathy was recognized therefore immune deficiency syndrome as well as HIV, and other viral infection were evaluated and excluded. Due to unusual clinical manifestations Hodgkin lymphomas was also explored and excluded by bronchoscopy and PET scan. Hydroxychloroquine (200 mg/day), kaletra (Lopinavir/ritonavir) (200 mg/day), azithromycin (250 mg/day), ceftriaxone 1 gr IV, and enoxaparin 40 mg/daywere administered. Subsequently heparin 5000 IU was added as a therapy. Despite lung involvement the patient never complained of dyspnea and oxygen saturation was always more than 94% with oxygen supply of 6 L/min. The patient developed leukopenia 2.8x10^9 (normal range 4-11 x10^9 /L). Despite all treatment, lung infiltration increased, patient felt weak and fatigue and oxygen saturation dropped to 88% within 24 hours therefore DSC therapy was considered as only choice for the patient. The patient received two doses of DSC, three and seven days after hospitalization. After the second dose of DSC, he could walk and had great improvement in clinical manifestations. The control CT scan was done two days after the second DSC infusion and showed clearance of infiltration and opacities (FIG. 2B). The IL-6, CRP, and CCL2 decreased and Oxygen saturation recovered to more than 97% (FIG. 6). The lymphadenopathy was completely resolved. The patient was discharged from the hospital three days after the second DSC dose.

### Patients 4 (UPN 9904)

A 56-year-old man with a past medical history of epilepsy/convulsion, and alcohol consumption for three years was admitted to the hospital. The patient discontinued anti epilepsy medications by himself and was not under anti-convulsion coverage. The patient was admitted with main complain of angina pectoris with ST elevation, dyspnea, respiratory distress, and decreased consciousness. Covid-19 infection symptoms started two weeks before admission, but the patient refused to refer to the hospital and after worsening of dry cough and chest pain referred to the hospital. At admission, oxygen supply was applied 10 L/min by face mask, but dyspnea worsens, and he developed myocardial infarction during CT scan imaging. The patient lost consciousness developed convulsion and subsequently he was intubated and transferred to the ICU. While in the ventilator the oxygen saturation was 95%. The patient experienced convulsions several times and was treated with anticonvulsive medication as well as supportive therapy for myocardial infarction. Drugs included were hydroxychloroquine (400 mg/day), meropenem (3 g/day), clopidogrel 150 mg/day, vancomycin 1500 mg/day, paracetamol 1 gr, pantoprazole 40 mg, sodium valproate 800 mg/day, phenytoin 100 mg/TDS, diltiazem 60 mg/TDS, and some other supportive medication were given. Despite all medication and supportive care, the patient became worse and his convulsions were not controllable. Therefore, two days after admission to the ICU, the patients received two doses of DSC therapy with three-day interval. The patient died nine days after admission to the hospital due to multi organ failure. However, the inflammatory markers IL-6, G-CSF, CRP and CCL-2 (FIG. 6) decreased after two doses of DSC therapy.

### Patients 5 (UPN 9905)

A 51-year-old man with diabetes mellitus (DM) since 12 years and congestive heart failure (CHF) was admitted to the emergency unit, with fever, dyspnea, fatigue, anorexia, and diarrhea. Clinical symptoms of Covid-19 infection started from 14 days ago. At admission oxygen saturation was 77% and therefore oxygen supply of 6 L/min through face mask was applied. At admission, a CT scan showed involvement of more than 50% of the lungs with focal ground glass infiltration. The patient received hydroxychloroquine 400 mg/day, enoxaparin 40 mg/day, azithromycin 250 mg/day, levofloxacin 750 mg/day, anticoagulant and an adjusted dose of Insulin. Despite treatment and supportive care dyspnea became worse and 6 L/min nasal oxygen was increased to 10 L/min oxygen via face mask. Three days after patient admission, dyspnea became even worse and the patient was treated with reserve bag with 10 L/min while blood oxygen level was 88%. Considering serious co-morbidity (DM) and uncontrolled diabetes mellitus which prohibited administration of corticosteroids, DSC therapy considered as choice for the patients and single dose of DSC was infused three days after hospitalization. 24 hours after DSC infusion blood level of oxygen reached to 90% whit an oxygen supply decreased to 4 L/min. Cytokines normalized after DSC infusion (FIG. 6). Two days after cell infusion dyspnea disappeared and the patient could walk and was discharged from the ICU to internal medicine ward. 24 hours later oxygen saturation was more than 99%, without oxygen supply and the patient was discharged from the hospital.

### Patients 6 (UPN 9906)

A 49-year-old man with a past medical history of diabetes mellitus (DM) and hypertension (HTN) for more than ten years was admitted to the hospital. Since a week, the patient had developed fever, shivering, abdominal pain, diarrhea, myalgia, cough, and dyspnea. A lung CT scan showed diffuse bilateral ground glass infiltration in the lungs. Oxygen saturation at admission was 74% (without mask) and oxygen supply of 10 L/min was applied for the patient using face mask. Beside the medication for his underlying disease, the patient received dexamethasone 8 mg/IV, atazanavir 300 mg/day, azithromycin 250 mg/day, enoxaparin 40 mg/day, ceftriaxone 2 g/IV and imipenem 3 g/day. Despite these medications and all supportive care oxygen saturation did not improved therefore face mask was shifted to reserve bag with 15-20 L/min. Even with reserve bag and high oxygen supply the oxygen saturation barely reached 78% (three days after admission), therefore DSC therapy was considered as the only chance for this patient. The first DSC infusion was done three days after admission. Patient O2 saturation stabilized, no dramatic improvement was seen, and the second DSC dose was infused three days later. After the second DSC dose, oxygen saturation increased and reached 92%. The General condition of the patient improved, and a recovery process started. Dyspnea decreased and cough decreased, and the patients could walk at the ward and the cytokines decreased in plasma following DSC infusion (FIG. 6). Due to improved clinical status, the physicians decided to infuse the third dose of DSC 9 days after admission. 48 hours later the patient was discharged from the hospital with normal oxygen saturation (>95%) and no dyspnea.

### Patients 7 (UPN 9907)

A 38-year-young man was admitted to the emergency with dyspnea, fever, and cough. Clinical symptom started three days ago, and patient was generally healthy with no past medical history. Oxygen saturation was 78% (without face mask) therefore oxygen supply of 8 L/min was ordered for the patients. Treatment with hydroxychloroquine 400 mg/day, atazanavir 200 mg/day, meropenem 3 gr/day, vancomycin 2 gr/day, enoxaparin 40 mg/day and methylprednisolone 50 mg/day started for the patient. Oxygen saturation did change so much and still low (without mask 81%), but IL-6 level was high, and patient had dyspnea. Three days after admission and due to no progress in patient’s situation and lung feature in CT scan the patient considered as DSC therapy candidate.

First dose of DSC was injected three days after admission and the second dose six days after admission. 24 hours after first and second DSC infusion the oxygen saturation reached to 87% and 93% respectively. Thereafter patient could keep oxygen level more than 98% (FIG. 6) without facemask, however face mask was always available. IL-6 level decreased after DSC therapy (FIG. 6). Six days after cell therapy patient was discharged from the hospital. Patients 8 (UPN 9908)

A 44-year-old woman with known history of hypertension (HTN) was admitted to the hospital with myalgia, fever, shivering, dry cough, and dyspnea started ten days ago. Preadmission CT scan showed multifocal ground glass bilateral infiltration of the lungs. Due to dyspnea and low oxygen saturation (82% without mask), oxygen supply 10 L/min started for the patient with reserve bag. After admission patient received dexamethasone 16 mg/day, enoxaparin 60 mg/day, and Sofosbuvir (400 mg/day), daclatasvir 60 mg/day. Patient received interferon beta 12x10⁶ SC every other day. Despite medication and oxygen supply (10 L/min) blood oxygen saturation did not improve much and still was 85%. Lung involvements worsen and oxygen supply shifted from face mask to reserve bag (10 L/min) but oxygen saturation became worse 80%. Thereafter to improve oxygen supply reserve bag changed to BiPAP. Since no improvement happened in patient situation, she was considered for cell therapy. First dose of DSC was infused to the patients five days after admission. 24 hours after cell infusion patient’s situation became stable and 48 hours after DSC infusion oxygen saturation reached to 95% (without a mask). Therefore, BiPAP shifted back to reserve bag (10 L/min). The patient could lie down and start eating food. Dyspnea improved and cough intensity decreased therefore patient transferred from the ICU to normal ward. Then oxygen supply changed to nasal tube (7 L/min). The patient was discharged in good health, 19 days after admission to the hospital. Patients 9 (UPN 9909)

A 50-year-old man known case of diabetes mellitus (DM) and hyperlipoproteinemia (HLP) admitted to the hospital with myalgia, cough and dyspnea started three days ago. Oxygen support immediately placed for the patients (10 L/min) and O2 saturation at admission time was 88% and 74%, with and without face mask, respectively. Treatment with sofosbuvir (400 mg/day), daclatasvir (60 mg/day) started. Moreover, the patient received interferon beta 12×10⁶ SC every other day, azithromycin 250 mg, ceftriaxone 2 gr/day, dexamethasone 16 mg/day, enoxaparin 60 mg/day. Patient was restless and was not cooperative with medical staff. Patients' situation became worse and four days after admission and with all supportive care oxygen level reached 89% and 65% with and without mask, respectively. To improve oxygenation face mask upgraded to reserve bag. CT scan indicated ground glass opacities, multifocal and bilateral. Five days after admission and despite all medications, the respiratory distress worsen, LDH level increased and patient situation was not stabilized. Consequently, the first dose of DSC was infused. 24 hours after DSC infusion oxygen level was stabilized between 89-92%. For the patient BiPAP was applied to keep better oxygenation. Three days later second dose of DSC was infused. After two dose DSC infusions patients general condition became stable and oxygen saturation reached to over 80%-88%. But the patient was agitated, irritable, and had very poor cooperation with staff and insisted to discharge from the hospital. Psychologic consulting was not effective and finally he discharged from the hospital by his own decision despite the high risk of mortality. Unfortunately, the patient did not have any compliance and cooperative with medical staff and his family and stopped all necessary medical cares, by his decision. Two days later he died at home due to cardiac arrest.

### Patients 10 (UPN 9910)

A 63-year-old woman with history of breast cancer and mastectomy admitted to the hospital with dyspnea and fever started seven days ago. At admission, the O2 saturation was 78% (without mask) therefore oxygen supply with 10 L/min started immediately. In lung CT scan 79% of both lungs were involved with multi focal infiltration. She also had mediastinal lymphadenopathy. Interferon beta 12×10⁶ SC every other day, ceftriaxone 2 gr/day, dexamethasone 16 mg/day started along with other supportive care in ICU. Oxygen saturation decreased by time and after three days face mask upgraded to reserve bag with 12 L/min oxygen supply. However, blood oxygen level deteriorated and hardly reached to 69% even with reserve bag. Four days after conventional treatment, CT scan showed the opacities in the lungs increased, LDH level elevated and patient’s situation worsen, therefore DSC therapy was requested for the patient. Five days after admission one dose of DSC were infused. 24 hours after DSC infusion oxygen saturation reached to 84% (without mask), patient situation became stable and disease progress stopped. Three days after DSC therapy oxygen saturation improved and stabilized over 88% (without mask) and control CT scan (three days after DSC treatment) indicated decrement in opacities and decreased of lung infiltration. Accordingly, the patients discharged from ICU and moved to ward. Thereafter oxygen supply decreased to 5 L/min and face mask changed to nasal tube. Oxygen supply without mask reached to 89%. Eight days after single DSC treatment patients discharged from the hospital with good health and need to any oxygen supply.

### The Effect of DSC Therapy on The Inflammatory Cytokines

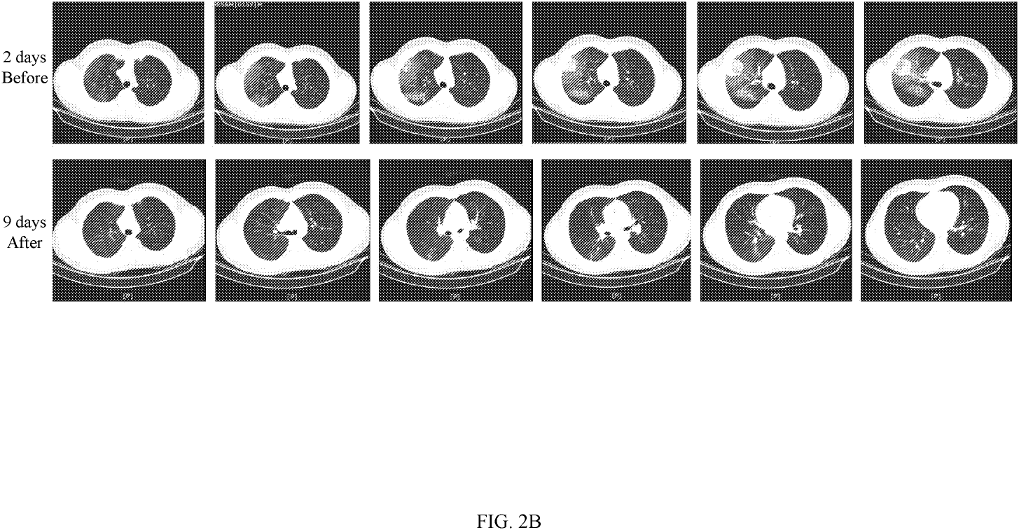

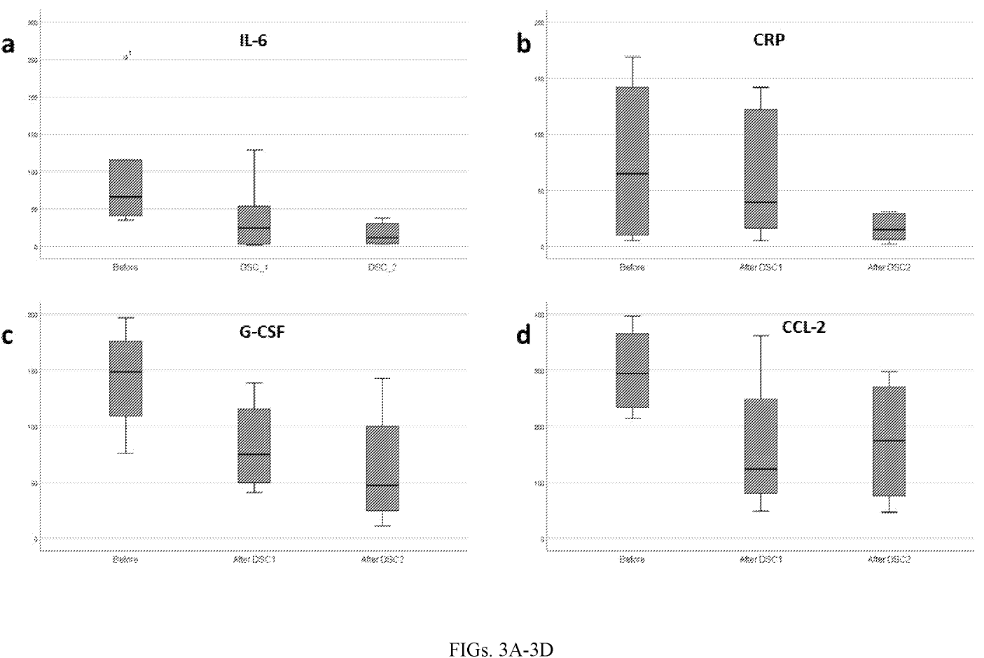

Cytokine storm and elevation of acute inflammatory mediators is a crucial manifest of ARDS following Covid-19 infection. IL-6, CRP, CCL-2 and G-CSF as acute inflammatory mediators were measured at different time points before and after DSC therapy in 7 out of 10 patients. FIG. 3 shows that DSC infusion decrease the level of these mediators and this decrement was demonstrated in clinical manifestation of patients. The median of IL-6 before DSC therapy was 69.3 pg/ml while it decreased to 11 pg/ml after 2 doses of DSC infusion (P<0.028) (FIG. 3A). Plasma level of CRP before and after DSC therapy was 57 and 13.5 mg/L, respectively (P<0.028) (FIG. 3B). the similar decreasing pattern was observed in the serum level of GCSF and CCL2, the levels of these cytokines decreased in five out of seven patients (FIGS. 3C-3D).

### The Effect of DSC Therapy on Oxygen Saturation

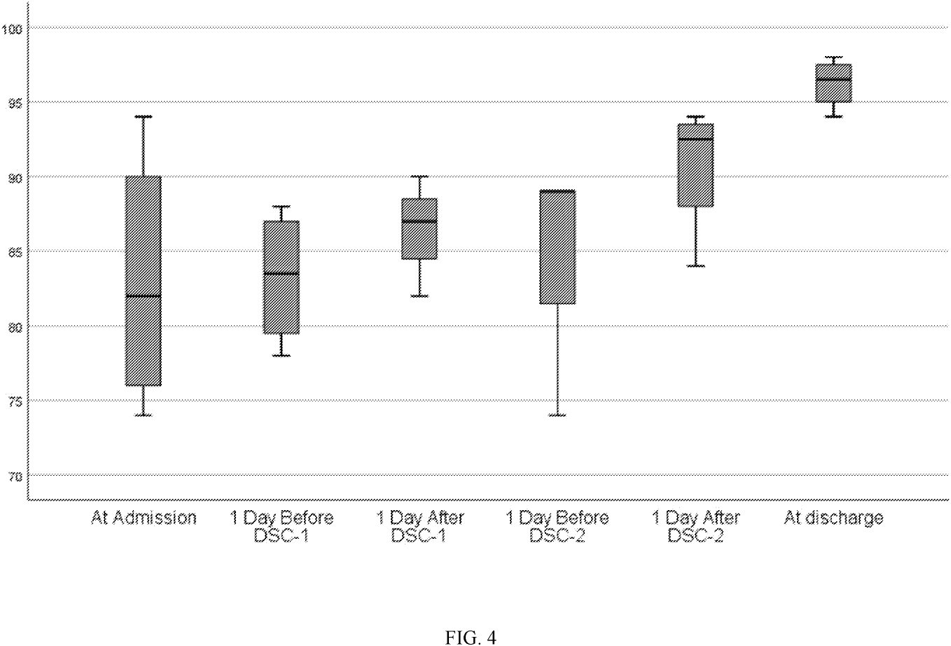

Blood oxygen saturation is an important prognostic factor in ARDS. In the present study we have noticed that immediate and durable effect of DSC infusion in increasing of blood oxygenation (FIG. 4, FIG. 6). As shown FIG. 6 the median of blood oxygen saturation before DSC therapy was 80.5%. DSC treatment significantly increased this level to 95% (P<0.012). More importantly DSC infusion a rapid effect on elevating blood oxygen level. FIG. 4 shows the dynamism of blood oxygen level before and after DSC infusion. 24 hours after each DSC infusion oxygen saturation raise and finally after the full protocol it reached to the normal level 95% (P<0.028).

### Side Effect or Adverse Effect DSC Therapy on Oxygen Saturation

Short- and long-term side effect related top cell infusion was evaluated in all patients. Except transient elevation in systolic blood pressure in two patients, which were controlled without any medication or discontinuation of cell infusion, we did not observe any DSC infusion related toxicity or side effect.

### Discussion

Cytokine storm and subsequent ARDS are the most unpleasant outcome after Covid-19 infection. ARDS develops in 42% of patients presenting with COVID-19 pneumonia, and 61-81% of those requiring intensive care. The mortality of Covid-19 is mainly due to the development of ARDS and respiratory failure following cytokine storm. To decrease the mortality rate, our therapeutic strategy should target controlling cytokine storm and ARDS.

In the present study we have shown a dramatic decrease in cytokine levels following infusion of DSCs. IL-6, G-CSF, CRP and CCL2 all decreased following infusion of DSCs. This is in line with our finding in GVHD patients. Inflammatory mediators e.g. IL-6, CRP and chemokine ligand CCL-2 has pivotal role in initiation and promotion of ARDS following Covid-19 infection. This is in agreement with a previous case report where in addition to IL-6 and G-CSF, also MCP-1, IL-8 and TNF alpha decreased in serum following infusion of DSCs for ARDS. Despite the important role of IL-6 in initiation/promotion of cytokine storm, DSCs is certainly more effective in controlling the cytokine storm than to use anti-IL-6 monoclonal antibodies. In a randomized, double-blind, placebo-controlled trial Tocilizumab, anti-IL-6 antibody, was given to 141 Covid-19 induced ARDS patients. Results indicated that Tocilizumab is not effective while showed some harm compare to control group.

Blood level of oxygen has important effect on the prognosis and outcome of ARDS induced Covid-19. Xie et. al. has shown that hypoxemia was independently associated with in-hospital mortality (Xie, J., et al.,. Mayo Clin Proc, 2020. 95(6): p. 1138-1147) and higher SpO2 levels after oxygen supplementation were associated with reduced mortality independently of age and sex. In the present study we could see an increment in PO2 level both as a rapid response following DSC infusion and durable improvement in blood oxygen level. This was reflected in clinical status of the patients, and almost all treated patients felt less pressure on chest and less dyspnea after DSC infusion.

Lung involvement is seen in majority of ARDS induced Covid-19 patients mainly with ground glass opacity or consolidation in plain chest X ray or CT scan. Although changes in CT scan is not diagnostic tool but some believes that changes in CT scan reflect in the clinical course of disease and outcome in patients. In the present study of Covid-19 induced ARDS we could see a quick reversal of ground glass opacity or consolidation in CT images following DSC treatment in patients. Liu et al reported the in more than 50% of the patients pulmonary sequalae disappearance might occur up 3 weeks after discharge. Interestingly, in our findings we observed that vanishing of lung pathology in patients' CT scan happen in shorter time compared to Liu’s report. This could be due the healing capacity or regenerative power of stromal cells which is unique characteristic of cell therapy.

In the present study one dose of DSC was given to three patients and 6 patients received two doses and another patient got three doses. The number of required doses to respond depends on the severity of the disease. When DSCs were used to treat acute GVHD the number of doses needed to reverse the disease also varied. While some patients responded to a single dose, several patients received two or more doses of DSCs. In the present study complete response of ARDS also seems to differ among the patients. Some had early response following single dose of DSCs, while others required additional doses before complete resolution was seen. This might reflect the severity and magnitude of cytokine storm in Covid-19 induced ARDS among different patients.

It may be debated, which type of MSCs should be used to treat clinical ARDS. A randomized study was performed using BM-MSCs at a dose of 10×10⁶ cells/kg. In the Matthay study BM-MSC therapy did not result in a better outcome than was seen in the placebo group (Matthay, M.A., et al., Lancet Respir Med, 2019. 7(2): p. 154-162). A problem with that study was that cell viability was relatively low 36-85% compared to a cell viability of more than 95% for DSCs in the present study. The ten times higher cell dose could probably not compensate for the poor cell survival, which may reveal that the quality of the BM-MSC in that study. There are several cell-based therapies for Covid-19 induced ARDS patients. Meng et.al reported a randomized trial using umbilical cord blood MSC (UC-MSC) (Meng, F., et al., Signal Transduct Target Ther, 2020. 5(1): p. 172). They have shown a good safety profile for UC-MSC however there was not a considerable better efficacy in UC-MSC compare to control group. The mean of CRP and IL-6 level before cell therapy in their patients (Median of 2.98 (1.2-36.4)) was less than what we observed in our patients (Median of 57 (7.1-169)), meaning that DSC showed better efficacy compared to the CB-MSC in more severe patients. Adipose derived MSCs also failed in a small randomized ARDS trial. Compared to MSCs from bone marrow, UC or fat, DSC seems preferable for ARDS.

One of the most important aims in a pilot study of a new treatment modality is evaluating the side effects and any SAEs. Two patients died during the treatment, and one patient died because he deliberately refused all therapy and left the hospital. The first patient died due to cardiac arrest, and MOF was the cause of death in the second patient. The cardiac arrest occurred in a patient with known IHD who was affected by COVID19-induced ALI/ARDS. Despite an increase in PO₂ and significantly decreased levels of IL-6, CRP and GCSF, which demonstrated the efficacy of the DSC therapy, the patients died three days later. The patient who developed MOF had a history of epilepsy and convulsions. It is possible that the corona virus infection may have infected several other organs apart from the lungs, including the CNS, which may have led to MOF and death. This occurred despite decreased levels of almost all inflammatory mediators in this patient.

None of the patients experienced an SAE that was related to the DSCs infusions. Increased systolic blood pressure was observed in two patients when they received DSCs; however, blood pressure control was achieved without any medication or discontinuation of the DSC infusions. This is consistent with the previous experience from using DSCs. The safety of DSCs is also well established from animal data. Furthermore, a clinical toxicity study in patients treated for acute GVHD and hemorrhagic cystitis showed only three minor transfusion reactions in 40 patients treated with DSCs. MSCs and DSCs may not have completely comparable toxicity as there are some obvious differences. For example, DSCs have a stronger anticoagulation effect compared with MSCs from bone marrow. This may seem problematic in COVID-19 patients who have an increased risk of thromboembolism. However, none of the patients in this study developed thromboembolism following infusion of DSCs. This is consistent with the experience to date from using DSCs for acute GVHD and hemorrhagic cystitis. From this study, it seems that DSCs are also safe for administration to COVID19-infected patients with pulmonary disease.

Following the treatment of COVID-19 induced ALI/ARDS using DSCs, we observed no toxicity or DSC infusion-related adverse effects. DSC therapy decreased the levels of cytokines IL-6, G-CSF, CRP and CCL 2. There was an increase in oxygenation and reversal of pulmonary disease. Four patients could be discharged from the hospital after a few days. Two patients with COVID19-induced ARDS and additional serious medical problems died of cardiac arrest and MOF, respectively. We conclude that DSC therapy could reverse the cytokine storm and should preferably be given to patients with COVID-19 disease with ARDS.

In a pilot study, SAE’s are of utmost importance and the primary aim. There were some SAE’s like death by cardiac arrest in the first patient and death by multiorgan failure (MOF)in another patient. However, the cardiac arrest happened in a patient with known IHD, who was affected by Covid-19 induced ALI/ARDS. Cardiac arrest occurred 3 days after the first DSC dose was given and despite an increase in PO2. The patient with MOF had epilepsy and convulsions and although he had a history of these disorders it is possible that Coronavirus may has infected several other organs apart from the lung including CNS and leading to MOF and death. This happened despite decreased cytokines and a probable reversal of the Covid-19 induced cytokine storm in the lung. None of the patients experienced an SAE that was associated with DSC infusion. This is in accordance with the previous experience using MSCs. Two multicenter studies demonstrated the safety of MSCs. MSCs have a well-documented low toxicity. The safety of DSCs is also well established from animal data. Furthermore, a clinical toxicity study in patients treated for acute GVHD and hemorrhagic cystitis showed only three minor transfusion reactions in 40 patients treated with DSCs. MSCs and DSCs may not have completely comparable toxicity, because there are some obvious differences. For instance, DSCs have a stronger anticoagulation effect compared to MSCs from bone marrow. This may seem problematic in Covid-19 patients, who have an increased risk for thromboembolism. None of the patients in this study developed thromboembolism following infusion of DSCs. This is in agreement with the experience, so far using DSCs for acute GVHD and hemorrhagic cystitis. Advantages of DSCs compared to BM-MSC from a safety aspect may be that DSCs are only half the size. From this study it seems that DSCs are also safe to infuse to Covid-19 infected patients with pulmonary disease. The rationale to use MSCs to treat ALI/ARDS is because these cells have a profound immunosuppressive and anti-inflammatory effect. After iv infusion, large amounts of DSCs like MSCs home in the lung, before distribution to liver, spleen and subsequently to other organs. Indeed, in these 10 patients we could register a dramatic decrease in cytokine levels following infusion of DSCs. IL-6, G-CSF, CRP and CCL 2 all decreased significantly following infusion of DSCS. This is in agreement with a previous case report where in addition to IL-6 and G-CSF, also MCP-1, IL-8 and TNF alpha decreased in serum following infusion of DSCs for ARDS. To give DSCs to decrease the cytokine storm is certainly more effective than to use anti-IL-6 monoclonal antibodies, which just decrease one of the cytokines responsible for the cytokine storm and ARDS.Anti-IL-6 antibody Toclizumab was also given to treat Covid-19 induced ARDS. In the present study we could also see a subsequent increase in PO2 suggesting a rapid effect on oxygenation after DSC infusion with a proposed anti-inflammatory effect in the lung.

In the present study of Covid-19 induced ARDS with typical ground glass opacity in the lungs, we could see a reversal in patients. When DSCs were used to treat acute GVHD the dose needed to reverse the disease also varied. While some patients responded to a single dose, several patients received two or more doses of DSCs. For instance, a child with advanced grade IV acute GVHD required six doses for complete response to occur. In the present study complete response of ARDS also seemed to differ among the patients. Some had early response already following one dose of DSCs, while others seemed to require additional doses before complete resolution was seen. One or more DSCs doses may be given until resolution of ARDS is obtained.

In seven out of seven of the patients the decrease of IL-6, increased oxygenation and disappearance of pulmonary infiltrates resulted in a dramatic improvement of general well-being. Subsequently six of the patients could be discharged from the hospital following a few days. It is of course impossible to know the definitive outcome in these patients if DSCs were not given. Most probably resolution of pulmonary disease would not have been so fast. Two patients died, one from cardiac arrest and the other from MOF. Death occurred in these two patients despite reduction of cytokines and improvement in oxygenation. The cells home to the lung and reversed the cytokine storm and pulmonary disease, but probably without any major effects on the damage to other organs. MSCs have been used to treat myocardial infarction, but then local infusion to the coronary arteries may be required for healing to be seen. When the cells are given systemically, they are not expected to affect other organs than the lung where an acute inflammation with neutrophil accumulation is taking place.

It may be debated, which type of MSCs should be used to treat clinical ARDS. A randomized study was performed using BM-MSCs at a dose of 10×10⁶ cells/kg. In that study BM-MSC therapy did not result in a better outcome than was seen in the placebo group. A problem with that study was that cell viability was relatively low 36-85 % compared to a cell viability of more than 95% for DSCs in the present study. The ten times higher cell dose could probably not compensate for the poor cell survival, which may reveal that the quality of the BM-MSC in that study may have been suboptimal. Probably other differences between BM-MSCs and DSCs may be more important. DSCs have a stronger immunosuppressive effect than BM-MSC. This was seen in vitro in Mixed Lymphocyte Culture and also in vivo when treating acute GVHD in patients. DSC treated patients had a better response rate and better survival as opposed to patients treated with BM-MSCs.BM-MSCs have other properties, like differentiation to bone cartilage and fat and are more suitable in regenerative medicine than DSCs who have poor differentiation capacity. Adipose derived MSCs also failed in a small randomized ARDS trial. Compared to MSCs from bone marrow or fat, DSC seems preferable for ARDS. If other sources of MSCs from umbilical cord or full placenta are useful in this context remains to be elucidated.

The cytokine storm and subsequent ARDS is the most common cause of death by Covid-19. However, Covid-19 may disseminate to many organs like heart tissue, kidney, intestine and CNS. Because there is no effective therapy to prevent Corona virus replication, early therapy using DSCs to prevent the cytokine storm is necessary for successful therapy. This was obtained in the four patients, who could be discharged soon after DSC therapy was given. However, in the two patients with multiple problems apart from Covid-19 induced ARDS, like cardiac problem and MOF, DSC therapy was not successful. When performing a subsequent placebo-controlled randomized study in patients with Covid-19 induced ARDS inclusion should be restricted to patients without signs of dissemination to other organs than the lung.

Following treatment of Covid-19 induced ALI/ARDS using DSCs, we observed no toxicity. DSC decreased the cytokines IL-6, G-CSF, CRP and CCL2. There was an increase in oxygenation and reversal of pulmonary disease. Four patients could subsequently be discharged from the hospital following a few days. Two patients with Covid-19 induced ARDS and additional serious medical problems died of cardiac arrest and MOF, respectively. It is concluded that DSC therapy to reverse the cytokine storm should preferentially be given to patients with Covid-19 disease restricted to the lung.

The teachings of all patents, published applications and references cited herein are incorporated by reference in their entirety.

### ITEMS

1. A method for treating a coronavirus disease in a subject in need thereof, the method comprising administering to the subject a therapeutically effective amount of decidua stromal cells (DSCs).
2. The method of item 1, wherein the coronavirus disease is SARS, MERS or COVID-19.
3. The method of item 1, wherein the subject has acute respiratory distress syndrome (ARDS), acute lung injury (ALI), or both.
4. The method of item 1, wherein the subject has acute respiratory distress syndrome (ARDS).
5. The method of item 1, wherein the DSCs are infused to the subject intravenously or intratracheally.
6. The method of item 1, wherein the DSCs are infused to the subject intravenously.
7. The method of item 4, wherein the DSCs are infused to the subject within about 1-20 days after initiation of ARDS in the subject.
8. The method of item 4, wherein the DSCs are infused to the subject within about 3-168 hours after initiation of ARDS in the subject.
9. The method of item 4, wherein the DSCs are infused to the subject within 72 hours after initiation of ARDS in the subject.
10. The method of item 1, wherein two or more doses of the DSCs are administered to the subject once every two to seven days.
11. The method of item 1, wherein two or more doses of the DSCs are administered to the subject once every three or four days.
12. The method of item 1, wherein the DSCs are administered to the subject once every three days.
13. The method of item 1, wherein the DCSs are administered to the subject at a dose of 0.5-2×10⁶ cells/kg.
14. The method of item 1, wherein the DCSs are administered to the subject at a dose of 1-1.2×10⁶ cells/kg.
15. The method of item 1, wherein the DSCs are suspended in saline with about 2.5-10% human plasma, human albumin or human platelet lysate.
16. The method of item 1, wherein the DSCs are suspended in saline with about 5% human albumin.
17. The method of item 2, wherein the DCSs are suspended at about 2-5×10⁶ cells/ml.
18. The method of item 2, wherein the DCSs are suspended at 2×10⁶ cells/ml.
19. The method of item 1, wherein the DSCs do not express CD45, CD34, CD31 or HLA class II, or a combination thereof.
20. The method of item 1, wherein the DSCs do not express CD11b, CD19, CD45, CD34, CD31 or HLA class II, or a combination thereof.
21. The method of item 1, wherein the DSCs are allogeneic to the subject.
22. The method of any one of items 1-21, wherein the DSCs:
   a) express CD29, CD73, CD90, CD105, or a combination thereof;
   b) do not express CD1 1b, CD45 or CD34, or a combination thereof; or
   c) both a) and b).
23. The method of any one of items 1-21, wherein the DSCs:
   a) express CD29, CD73, CD90, CD105, PDL-1 or PDL-2, or a combination thereof;
   b) do not express CD1 1b, CD45 or CD34, or a combination thereof; or
   c) both a) and b).
24. The method of any one of items 1-23, wherein the DSCs has a cell viability of at least 85%.
25. The method of any one of items 1-23, wherein the DSCs has a cell viability of at least 90%.
26. The method of any one of items 1-25, wherein the subject is a human patient between about 6 months and 75 years old.
27. The method of any one of items 1-26, wherein the subject has no history of co-morbidity.
28. The method of any one of items 1-26, wherein the subject has a history of co-morbidity.
29. The method of any one of items 1-28, wherein the subject experiences Covid-19 induced cytokine storm in the lung.
30. The method of any one of items 1-29, wherein the subject develops limited or multiple pulmonary ground glass opacities or pulmonary infiltration, or both, optionally, in CT scan or other imaging method.
31. The method of any one of items 1-30, wherein prior to receiving DSCs, the subject has received anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.
32. The method of any one of items 1-31, wherein the therapeutically effective number of DSCs is sufficient to:
   a) reduce IL-6, G-CSF, CRP or CCL2, or a combination thereof;
   b) clear multiple pulmonary ground glass opacities or clear pulmonary infiltration, or both;
   c) increases blood oxygen saturation level, or a combination thereof.
33. The method of any one of items 1-32, further comprising administering to the subject a therapeutically effective amount of anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.
34. A method for treating a viral-induced acute respiratory distress syndrome (ARDS) in a subject in need thereof, the method comprising administering to the subject a therapeutically effective amount of decidua stromal cells (DSCs).
35. The method of item 34, wherein the virus is a coronavirus.
36. The method of item 35, wherein the coronavirus is SARS, MERS or COVID-19.
37. The method of item 34, wherein the DSCs are infused to the subject intravenously or intratracheally.
38. The method of item 34, wherein the DSCs are infused to the subject intravenously.
39. The method of item 38, wherein the DSCs are infused to the subject within about 1-20 days after initiation of ARDS in the subject.
40. The method of item 38, wherein the DSCs are infused to the subject within about 3-168 hours after initiation of ARDS in the subject.
41. The method of item 38, wherein the DSCs are infused to the subject within 72 hours after initiation of ARDS in the subject.
42. The method of item 34, wherein two or more doses of the DSCs are administered to the subject once every two to seven days.
43. The method of item 34, wherein two or more doses of the DSCs are administered to the subject once every three or four days.
44. The method of item 34, wherein the DSCs are administered to the subject once every three days.
45. The method of item 34, wherein the DCSs are administered to the subject at a dose of 0.5-2×10⁶ cells/kg.
46. The method of item 34, wherein the DCSs are administered to the subject at a dose of 1-1.2×10⁶ cells/kg.
47. The method of item 34, wherein the DSCs are suspended in saline with about 2.5-10% human plasma, human albumin or human platelet lysate.
48. The method of item 34, wherein the DSCs are suspended in saline with about 5% human albumin.
49. The method of item 35, wherein the DCSs are suspended at about 2-5×10⁶ cells/ml.
50. The method of item 35, wherein the DCSs are suspended at 2×10⁶ cells/ml.
51. The method of item 34, wherein the DSCs do not express CD45, CD34, CD31 or HLA class II, or a combination thereof.
52. The method of item 34, wherein the DSCs do not express CD1 1b, CD19, CD45, CD34, CD31 or HLA class II, or a combination thereof.
53. The method of item 34, wherein the DSCs are allogeneic to the subject.
54. The method of any one of items 34-53, wherein the DSCs:
   a) express CD29, CD73, CD90 or CD105, or a combination thereof;
   b) do not express CD11b, CD45 or CD34, or a combination thereof; or
   c) both a) and b).
55. The method of any one of items 34-53, wherein the DSCs:
   a) express CD29, CD73, CD90, CD105, PDL-1 or PDL-2, or a combination thereof;
   b) do not express CD11b, CD45 or CD34, or a combination thereof; or
   c) both a) and b).
56. The method of any one of items 34-55, wherein the DSCs has a cell viability of at least 85%.
57. The method of any one of items 34-55, wherein the DSCs has a cell viability of at least 90%.
58. The method of any one of items 34-57, wherein the subject is a human patient between about 6 months and 75 years old.
59. The method of any one of items 34-58, wherein the subject has no history of co-morbidity.
60. The method of any one of items 34-58, wherein the subject has a history of co-morbidity.
61. The method of any one of items 34-60, wherein the subject experiences Covid-19 induced cytokine storm in the lung.
62. The method of any one of items 34-61, wherein the subject develops limited or multiple pulmonary ground glass opacities or pulmonary infiltration, or both, optionally, in CT scan or other imaging method.
63. The method of any one of items 34-62, wherein prior to receiving DSCs, the subject has received anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.
64. The method of any one of items 34-63, wherein the therapeutically effective number of DSCs is sufficient to:
   a) reduce IL-6, G-CSF, CRP or CCL2, or a combination thereof;
   b) clear multiple pulmonary ground glass opacities or clear pulmonary infiltration, or both;
   c) increases blood oxygen saturation level, or a combination thereof.
65. The method of any one of items 34-64, further comprising administering to the subject a therapeutically effective amount of anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for treating a coronavirus disease in a subject in need thereof, the method comprising administering to the subject a therapeutically effective amount of decidua stromal cells (DSCs).

2. The method of claim 1, wherein the subject has acute respiratory distress syndrome (ARDS), acute lung injury (ALI), or both.

3. A method for treating a viral-induced acute respiratory distress syndrome (ARDS) in a subject in need thereof, the method comprising administering to the subject a therapeutically effective amount of decidua stromal cells (DSCs).

4. The method of claim 3, wherein the virus is a coronavirus.

5. The method of claim 1, wherein the coronavirus is SARS, MERS or COVID-19.

6. The method of claim 1, wherein the DSCs are infused to the subject intravenously or intratracheally, within about 1-20 days after initiation of ARDS in the subject.

7. The method of claim 1, wherein two or more doses of the DSCs are administered to the subject once every two to seven days.

8. The method of claim 1, wherein the DCSs are administered to the subject at a dose of 0.5-2 × 10⁶ cells/kg.

9. The method of claim 1, wherein the DSCs are suspended in saline with about 2.5-10% human plasma, human albumin or human platelet lysate.

10. The method of claim 1, wherein the DCSs are suspended at about 2-5 × 10⁶ cells/ml.

11. The method of claim 1, wherein the DSCs do not express CD11b, CD19, CD45, CD34, CD31 or HLA class II, or a combination thereof.

12. The method of claim 1, wherein the DSCs express CD29, CD73, CD90, CD105, PDL-1 or PDL-2, or a combination thereof.

13. The method of claim 1, wherein the DSCs has a cell viability of at least 85%.

14. The method of claim 1, wherein the subject is a human patient between about 6 months and 75 years old, and wherein the subject has no history of co-morbidity.

15. The method of claim 1, wherein the subject is a human patient between about 6 months and 75 years old, and wherein the subject has a history of co-morbidity.

16. The method of claim 1, wherein the subject experiences Covid-19 induced cytokine storm in the lung.

17. The method of claim 1, wherein the subject develops limited or multiple pulmonary ground glass opacities or pulmonary infiltration, or both, optionally, in CT scan or other imaging method.

18. The method of claim 1, wherein prior to receiving DSCs, the subject has received anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.

19. The method of claim 1, wherein the therapeutically effective number of DSCs is sufficient to:
a) reduce IL-6, G-CSF, CRP or CCL2, or a combination thereof;
b) clear multiple pulmonary ground glass opacities or clear pulmonary infiltration, or both;
c) increases blood oxygen saturation level, or
a combination thereof.

20. The method of claim 1, further comprising administering to the subject a therapeutically effective amount of anticoagulant, atazanavir, azithromycin, ceftriaxone, clopidogrel, daclatasvir, dexamethasone, enoxaparin, heparin, hydrocortisone, hydroxychloroquine, IFN B-1, imipenem, Interferon beta, IVIG, Kaletra® (Lopinavir/ritonavir), levofloxacin, meropenem, methylprednisolone, naproxen, oseltamivir, pantoprazole, paracetamol, Sofosbuvir or vancomycin, or a combination thereof.
